(19) United States
(12) Patent Application Publication
(10) Pub. No.: US 2021/0369882 A1
(43) Pub. Date: Dec. 2, 2021

(54) SYSTEMS AND METHODS FOR STERILIZING WASTE

(71) Applicant: Approved Storage & Waste Hauling, Mt. Vernon, NY (US)

(72) Inventor: Charles Dippolito, Jr., Mt. Vernon, NY (US)

(21) Appl. No.: 17/333,789

(22) Filed: May 28, 2021

Related U.S. Application Data
(60) Provisional application No. 63/032,135, filed on May 29, 2020.

(51) Int. Cl. A61L 2/07; A61L 2/26; A61L 11/00; B09B 3/00

(52) U.S. Cl. CPC A61L 2/07; A61L 2/26; A61L 11/00; A61L 2202/121; B09B 3/0091; A61L 2202/122; B09B 3/0075

(57) ABSTRACT

Disclosed herein is a receptacle configured to receive and contain waste, such as medical waste, during sterilization, wherein the receptacle is water permeable and heat resistant at a temperature of up to about 320° F. Also disclosed are systems and methods for sterilizing waste, such as with an autoclave, including the use of the receptacle.

### CROSS-REFERENCE TO RELATED APPLICATIONS

This application claims the benefit of and priority to U.S. Provisional Application Ser. No. 63/032,135, having the title “SYSTEMS AND METHODS FOR STERILIZING WASTE”, filed on May 29, 2020, the disclosure of which is incorporated herein by reference in its entirety.

### BACKGROUND

Commercially available bags used for autoclave purposes may stick to the bin or can or autoclave apparatus, or cause bins or cans to adhere to each other during or after the autoclave cycle.

### SUMMARY

Embodiments of the present disclosure provide for systems for sterilizing waste, methods for sterilizing waste, methods of use, and the like.

An embodiment of the present disclosure includes a system for sterilizing waste. The method can include a receptacle configured to receive and contain the waste during sterilization, wherein the receptacle is water permeable and heat resistant at a temperature of up to about 320° F.

An embodiment of the present disclosure includes a method of sterilizing waste, which can include providing a receptacle, wherein the receptacle is water permeable and heat resistant to a temperature of up to about 285° F. and filling the receptacle with waste. The method can further include autoclaving both the receptacle and the waste then cooling the autoclaved receptacle and waste.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows an example of a receptacle used as a bin liner lining an autoclave cart according to embodiments of the present disclosure.

FIG. 2A shows a receptacle used as a bin liner lining an autoclave cart, where the cart has been filled with medical waste, according to embodiments of the present disclosure. FIG. 2B shows a receptacle used as a bin liner lining an autoclave cart, where the cart has been filled with medical waste in bags and the bin liner has been tied closed, according to embodiments of the present disclosure.

FIG. 3 shows a receptacle used as a bin liner with waste after having been autoclaved, illustrating the prevention of debris adhesion to the cart sides.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Disclosed herein are devices, systems, and methods for sterilizing medical waste using an autoclave process. More specifically, the disclosed devices, systems, and methods include at least partially enclosing the medical waste in a bin liner, such as a nonwoven textile bag. The bin liner may be disposed in another container, such as a metal bin, and inserted into the autoclave apparatus, where steam may be introduced to sterilize the contents of the autoclave. The bin liner does not melt under the autoclave processing conditions, and is water permeable so that it does not retain significant amounts of vapor or liquid resulting from the autoclave process.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed devices, systems and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. Definitions

A “textile” may be defined as any material manufactured from fibers, filaments, or yarns characterized by flexibility, fineness, and a high ratio of length to thickness. Textiles generally fall into two categories. The first category includes textiles produced directly from webs of filaments or fibers by randomly interlocking to construct non-woven fabrics and felts. The second category includes textiles formed through a mechanical manipulation of yarn, thereby producing a woven fabric, a knitted fabric, a braided fabric, a crocheted fabric, and the like.

A “nonwoven” textile, as used herein, refers to a textile material that is made from fibers that are bonded together (as opposed to woven or knitted textiles). The fibers can include long (e.g., continuous extruded) or short staple fibers, or a combination thereof. The fibers in the material can be bonded together by chemical, mechanical, heat, sonic, or solvent treatment, as can be envisioned by one of ordinary skill in the art. The material can be manufactured using any of a variety of processes, including but not limited to carding, air lay, wet lay, spun bond, melt-blown, spunlaid, flashspun, staple nonwoven, and combinations thereof, e.g., spunbond+meltblown+spunbond (SMS).

An “autoclave” as used herein refers to a device or apparatus used to carry out industrial and scientific processes requiring elevated temperature and pressure in relation to ambient pressure/temperature. Autoclaves are used in various applications, including medical applications to perform sterilization and in the chemical industry to cure coatings and vulcanize rubber and for hydrothermal synthesis.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise.

It should be noted that measurements and numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### B. Systems and Apparatus for Sterilizing Waste

In one aspect, the disclosure relates to systems and apparatus for sterilizing waste in an autoclave process. More specifically, in one aspect, the present disclosure relates to systems for sterilizing waste, including a receptacle configured to contain waste therein during an autoclave sterilization process. The term “receptacle” can also be referred to as a bin liner, bag, or container. In some aspects, the receptacle can be used with or without another receptacle. For example, the receptacle may be used as a bin liner disposed in a metal or stainless steel bin used to transport the waste material before, during, and/or after the autoclave process, referred to as an autoclave bin liner. As used herein, the term “bin” can refer to an autoclave cart, autoclave chamber, rack system for an autoclave chamber, and the like.

According to various aspects, the disclosed receptacle is suitable for use under autoclave process conditions. Autoclave processes use heat, pressure, and/or steam to treat and sterilize waste materials. In certain aspects, the receptacle is heat resistant to autoclave temperatures of up to about 320° F., such as temperatures of 270° F., 280° F., 285° F. or greater.

Advantageously, the receptacles disclosed herein are sufficiently heat resistant, to prevent the receptacle from melting during autoclaving process. This prevents the receptacle as well as its contents from adhering to the autoclave bin. Commercially-available bin liners such as those used in industrial medical waste autoclaves are typically made from films, such as polypropylene films. These film bin liners tend to melt to the autoclave bins, and/or allow the waste material inside to adhere to the sides of the autoclave bin. Such adhesion of the liner and/or the waste can result in expensive regular clean outs to remove the melted material from the sides, additional personnel hours to remove the material, and/or reduce the efficiency and capacity of the autoclave. In comparison, the disclosed receptacles do not melt during the typical autoclave process, avoiding the additional resources and costs associated with cleaning the receptacles and/or autoclave.

Traditionally, one method of avoiding the adhesion of the commercial bin liners and/or the waste to the bin or the autoclave chamber has been the use of polytetrafluoroethylene (PTFE or Teflon®) coating of the autoclave bins and/or chambers. However, PTFE materials are costly, and need to be re-applied on a regular basis. The use of the disclosed receptacles can reduce the need for PTFE coating of the autoclave bins or chambers.

The disclosed receptacles are permeable to water and water vapor, allowing for waste to be contained, but for water and/or steam to pass through the receptacle. Conventional film bin liners can trap and contain steam and/or water generated during the autoclave process, which adds unnecessary water weight to be handled in downstream processing. In comparison, because the disclosed receptacles are water permeable, process steam and/or water can drain from the receptacle, resulting in a lighter bag at the conclusion of the autoclave process. This can improve the cooling of the receptacle. In addition, the reduced water weight adds less strain to the receptacle when it is removed from the autoclave bin. The lower weight lessens the likelihood of the liner tearing and allowing melted waste to adhere to the autoclave bin. Additionally, the reduced weight can result in reduced load on downstream processing equipment, and can reduce handling costs, since waste disposal costs are often determined at least in part by weight.

According to various aspects, the described receptacles comprise a textile, more specifically a nonwoven textile. The nonwoven textile may comprise one or more textile layers. The nonwoven textile or layer thereof can comprise continuous fibers (e.g., spunbond or meltblown textiles), staple fibers (e.g., wet-laid or dry-laid textiles), or a combination thereof (e.g., a spunbond-meltblown-spunbond textile). The fibers of the nonwoven textile or layer may be entangled, and bonded by any of a variety of techniques such as mechanical bonding, chemical bonding, resin bonding, thermal bonding, sonic bonding, or a combination thereof. In various aspects, the receptacle comprises a spunbond nonwoven textile.

The nonwoven textile can comprise any of a variety of fibers that provide the necessary or desired characteristic described herein. The fibers can be natural or synthetic, or a combination thereof. In various aspects, at least the outer layer of the nonwoven textile comprises a material having a melting point that is higher than the temperature reached during the autoclave sterilization process. Examples of materials for the outer layer of the nonwoven textile include polypropylene (PP), polyamide (PA), polyethylene terephthalate (PET), silicon, polyvinylidene fluoride (PVDF), such as polytetrafluoroethylene (PTFE), polyaramid, polyimide, polyetherimide, and the like, including combinations thereof. In some aspects, the nonwoven textile comprises polypropylene fibers.

In various aspects, the disclosed receptacle has sufficient strength to support the weight of the waste disposed therein. For example, the strength of the receptacle can be determined on the basis of one or more of basis weight, tear strength, tensile strength, and/or other properties associated with the receptacle.

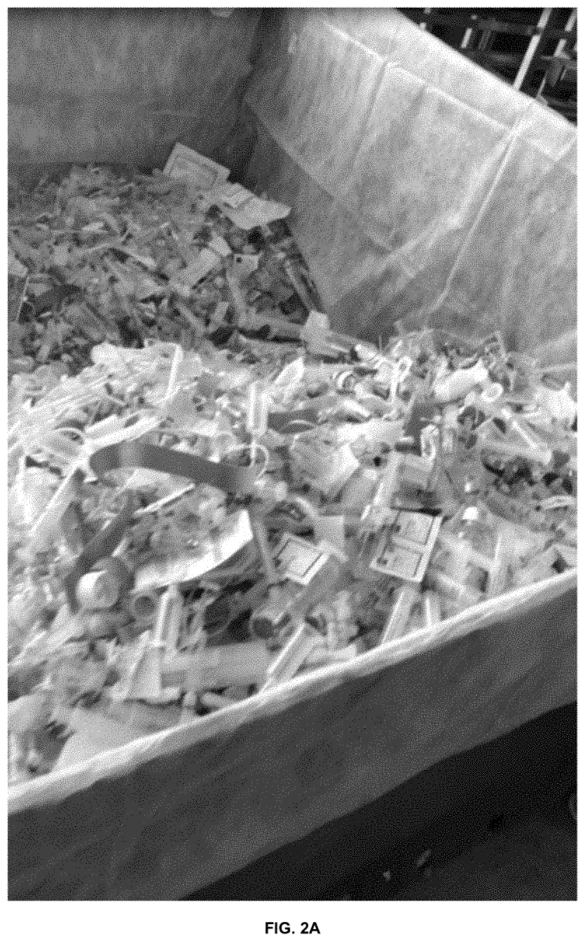

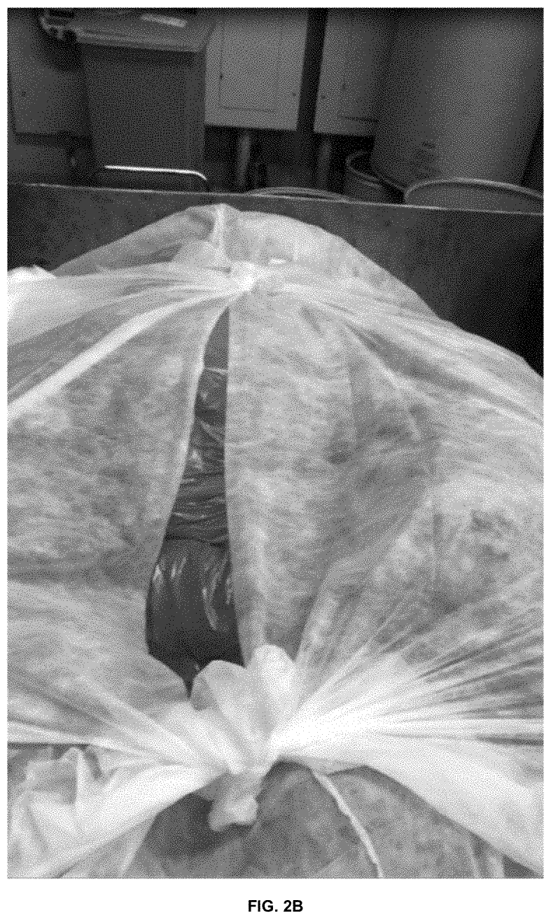

In various aspects, the receptacle can optionally comprise a closure mechanism, so that the receptacle may at least partially enclose the waste disposed therein (FIG. 2B). In some aspects, the closure may be integral with the receptacle, such as excess material or flaps that are part of the receptacle. In some aspects, the closure may be a separate component that is coupled with the receptacle, such as a drawstring, a zipper, a tie, or other closure means.

In some aspects, the receptacle can optionally include printing, such as biohazard symbols, company logos, or writing. The receptacle can also optionally include heat and/or steam indicators or thermochromic labels to indicate whether the waste inside has been autoclaved.

As can be envisioned by one of ordinary skill in the art, the receptacle can be sized for use in various types of autoclave systems, from large industrial systems having capacities of up to about 950 pounds per can, our up to about 3,800 pounds per cycle, to benchtop autoclaves in labs or medical offices. While the systems, methods, and materials described herein refer to use in medical waste, said methods and materials can also be used in other settings in which high heat and pressure may be used, such as in laboratory or clinical autoclaves. The systems, methods, and materials described herein may also have uses in other industries utilizing steam, such as oil and gas.

### C. Methods of Sterilizing Waste

In one aspect, the disclosure relates to methods for sterilizing waste in an autoclave apparatus. An exemplary autoclave uses high pressure steam to sterilize material placed therein. An exemplary autoclave apparatus includes an interior autoclave chamber, and may include a steam generating system to generate saturated steam to be delivered into the autoclave, and a controller to ensure that the autoclave operates as the correct temperature and pressure. A range of suitable steam temperatures and pressures to be achieved in the autoclave are from about 180° F. to about 320° F. (about 82° C. to about 160° C.) and from about 1 to about 10 bar (14.5 to 145.0 psi) respectively. The autoclave apparatus may also include a steam/water collection system.

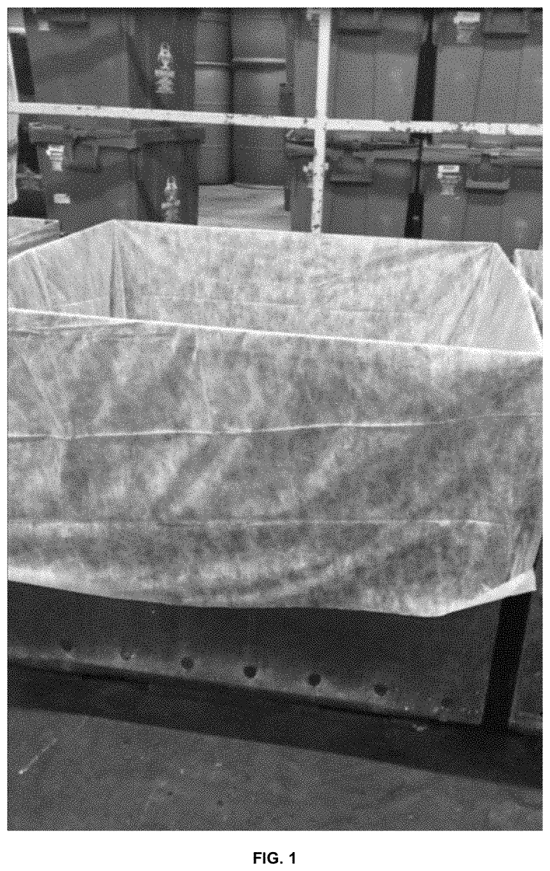

An exemplary autoclave has a capacity to receive multiple autoclave bins within the interior autoclave chamber. Each bin is a metal receptacle configured to receive and hold a certain amount of waste material, and to withstand the autoclave process conditions. The method may include inserting a receptacle as described above into an autoclave bin, to provide a bin liner (FIG. 1), and then disposing the waste material inside the bin liner (FIGS. 2A-2B). The bin liner may be tied or otherwise closed to at least partially enclose the waste material.

The method may further include loading one or more autoclave bins inside the chamber of the autoclave. Once the bins are inside the autoclave chamber, the doors of the autoclave chamber are closed and secured. Optionally, a vacuum may be drawn in the chamber, to assist with steam penetration. The steam generating system will then provide the required steam to the interior of the autoclave chamber to heat the autoclave to the desired temperature, for example 136° C. (277° F.), a desirable pressure, for example 8 bar (116 psi), or both. The temperature and/or pressure may be determined based on the necessary or desired sterilization results. For example, the autoclave chamber may be heated to a temperature of greater than about 180° F., or greater than about 185° F., or greater than about 190° F., or greater than about 195° F., or greater than about 200° F., or greater than about 205° F., or greater than about 210° F., or greater than about, 215° F., or greater than about 220° F., or greater than about 225° F., or greater than about 230° F., or greater than about 235° F., or greater than about 240° F., or greater than about 245° F., or greater than about 250° F., or greater than about 255° F., or greater than about 260° F., or greater than about 265° F., or greater than about 270° F., or greater than about 275° F., or greater than about 280° F., or greater than about 285° F., or greater than about 290° F., or greater than about 295° F., or greater than about 300° F., or greater than about 305° F., or greater than about 310° F., or greater than about 315° F., as necessary or desired. The pressure within the autoclave chamber may be increased to a pressure of greater than about 1 bar, or greater than about 2 bar, or greater than about 3 bar, or greater than about 4 bar, or greater than about 5 bar or greater than about 6 bar, or greater than about 7 bar, or greater than about 8 bar, or greater than about 9 bar, as necessary or desired.

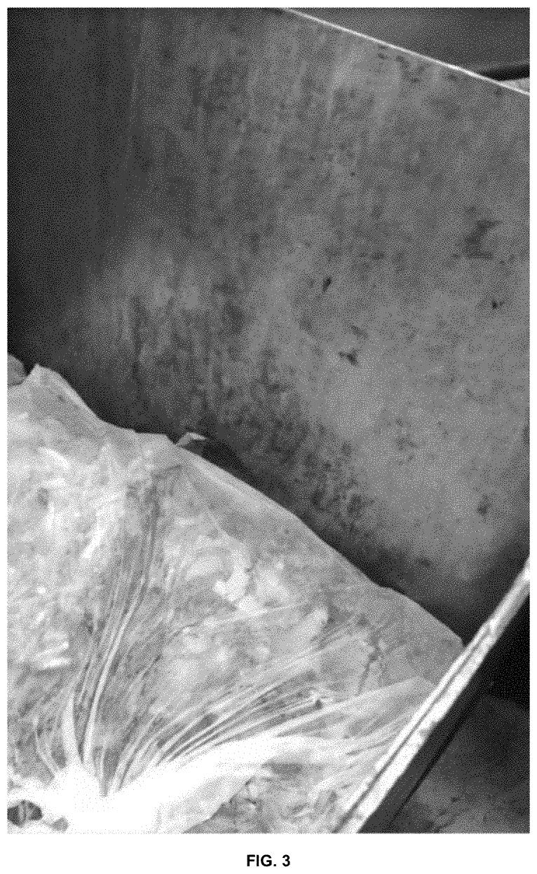

The temperature and pressure may be controlled and maintained for a predetermined period of time to obtain the necessary or desired sterilization. In some aspects, the sterilization process may be repeated for multiple cycles (e.g., 2-3 cycles) of steam injection followed by drawing a vacuum. Once the sterilization cycle or cycles are completed, the steam is removed from the autoclave and the waste materials in the autoclave bins is allowed to cool. Once cool, the autoclave bins are removed from the autoclave chamber. The bin liner may prevent the sterilized waste from adhering to the autoclave bin, as shown in FIG. 3. The bin liners and sterilized waste materials may be removed from the autoclave bins. The sterilized waste materials may then be further processed and/or disposed. For example, the waste can then be dumped into a larger container for further processing and/or disposal.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A system for sterilizing waste, comprising a receptacle configured to receive and contain the waste during sterilization, wherein the receptacle is water permeable and heat resistant at a temperature of up to about 320° F.

2. The system of claim 1, wherein the receptacle is a nonwoven textile having one or more layers selected from carded, air laid, wet laid, spun bond, melt-blown, spunlaid, flashspun, staple nonwoven, spunbond+meltblown+spunbond (SMS), or combinations thereof.

3. The system of claim 2, wherein the nonwoven textile comprises at least one layer of polypropylene.

4. The system of claim 1, wherein the receptacle further comprises a closure, wherein the closure is selected from a flap, a drawstring, or a tie.

5. The system of claim 1, wherein the system further comprises an autoclave and an autoclave bin, and the receptacle is a liner for the autoclave bin.

6. The system of claim 5, wherein the receptacle does not adhere to the autoclave bin after autoclaving.

7. A method of sterilizing waste, comprising the steps of:
a) providing a receptacle, wherein the receptacle is water permeable and heat resistant to a temperature of up to about 320° F.;
b) filling the receptacle with waste;
c) autoclaving the receptacle and the waste; and
d) cooling the autoclaved receptacle and the waste.

8. The method of claim 7, further comprising disposing the receptacle at least partially inside an autoclave bin, to provide an autoclave bin liner.

9. The method of claim 7, further comprising at least partially closing the receptacle.

10. The method of claim 7, further comprising disposing of the receptacle and the autoclaved waste.

11. The method of claim 7, wherein the receptacle is a nonwoven textile having one or more layers selected from carded, air laid, wet laid, spun bond, melt-blown, spunlaid, flashspun, staple nonwoven, spunbond+meltblown+spunbond (SMS), or combinations thereof.

12. The method of claim 7, wherein the receptacle comprises polypropylene fibers.

13. The method of claim 7, wherein the autoclaving comprises introducing steam to the receptacle and the waste.

14. The method of claim 13, wherein the autoclaving comprises increasing the temperature of the receptacle and the waste to a temperature of about 180° F. to about 320° F. (about 82° C. about 160° C.).

15. The method of claim 13, wherein the autoclaving comprises inserting the receptacle and the waste into an autoclave chamber, and performing one or more cycles comprising:
drawing a vacuum in the autoclave chamber enclosing the receptacle and the waste;
introducing steam to the autoclave chamber;
increasing the temperature within the autoclave chamber to a temperature of about 180° F. to about 320° F. (about 82° C. about 160° C.); and
increasing a pressure within the autoclave chamber to about 1 to about 10 bar (about 14.5 psi to about 145.0 psi).

16. The method of claim 8, wherein after the cooling, the receptacle does not adhere to the autoclave or the autoclave bin.
